(19) United States
(12) Patent Application Publication
(10) Pub. No.: US 2021/0391056 A1
(43) Pub. Date: Dec. 16, 2021

(54) HEALTH BIG DATA SERVICE METHOD AND SYSTEM BASED ON REMOTE FUNDUS SCREENING

(71) Applicant: FUZHOU YIYING HEALTH TECHNOLOGY CO., LTD., Fuzhou (CN)

(72) Inventors: LUN YU, Fuzhou (CN); YING-QIANG QIU, Fuzhou (CN); JIA-WEN LIN, Fuzhou (CN); XIN-RONG CAO, Fuzhou (CN); LI-NA WANG, Fuzhou (CN); Lin-Jie OU, Fuzhou (CN); Lan-Yan XUE, Fuzhou (CN)

(73) Assignee: FUZHOU YIYING HEALTH TECHNOLOGY CO., LTD., Fuzhou (CN)

(21) Appl. No.: 17/271,611

(22) PCT Filed: Dec. 27, 2018
(86) PCT No.: PCT/CN2018/124488

§ 371 Date: Sep. 8, 2021

(30) Foreign Application Priority Data
Aug. 31, 2018 (CN) 201811013848.2

(51) Int. Cl. G16H 30/20; G16H 50/20; G06T 7/00; A61B 3/12; A61B 3/00

(52) U.S. Cl. CPC G16H 30/20; G16H 50/20; G06T 7/0012; G06T 2207/30041; A61B 3/0025; A61B 3/0058; A61B 3/12

(57) ABSTRACT

Big data health service method and system based on remote fundus screening are provided. The method includes steps of: acquiring information to be analyzed sent by remote terminal agency; pre-interpreting information to be analyzed, and judging whether information to be analyzed is qualified; extracting characteristic data from information to be analyzed if it is qualified, and forming structured quantitative index; sorting and analyzing characteristic data and quantitative index according to knowledge calculation model to obtain analysis conclusion; and storing information to be analyzed, characteristic data, quantitative index, and analysis conclusion into pre-designed database. The above steps can produce quantitative index and characteristic data with uniform comparability for final fundus images such processed, no matter what type of fundus camera or which working mode is used, so that a whole big data service platform is established, and medical practitioners are facilitated greatly in disease diagnosis and the like.

### BACKGROUND OF THE INVENTION

### 1. Field of the Invention

The present disclosure relates to the field of big data on health, and more particularly, to a big data health service method and system based on remote fundus screening.

### 2. Description of the Prior Art

According to the latest publication of the World Health Organization, 3.477 billion people were diagnosed with diabetes worldwide today, this number will have exceeded 64 billion by the year 2040, and it is estimated that diabetic retinopathy (DR) has affected more than 100 billion people; according to the population and demographic structure, China has about 250 million hypertension patients (i.e., one in every three adults is a hypertension patient in China), accounting for about one-third of the hypertension patients globally, and the prevalence rate is increasing, especially in the aged people, while the controlled cases amount only to 5.7%. The number of diabetes patients in China has also exceeded 100 million. Diabetes and its complications produced a serious socio-economic burden.

However, we still lack an efficient early-warning or big screening platform for stroke, DR, DN, glaucoma, cataract and other major diseases or complications; it is difficult for mobile medical treatment to acquire accurate information on brain, heart, eye, kidney and other target organs, and to provide personalized health services.

The fundus camera technology for diabetic retinopathy (DR) screening has matured, however, different types of fundus cameras and their different modes of operation lead to different sizes, resolutions, structures, and the like of the acquired fundus images. Images of the same eye of the same user can even be different if they are collected by different devices or at different times, it is thus impossible to compare and analyze quantitatively the images from multiple examinations of an individual as per indexes due to different views and resolutions, and it is also difficult to analyze quantitatively, gather statistics and compare the retinopathy syndromes, positions, sizes or vascular changes of fundus images collected from different people, or from the same person but at different times or with different devices. This affects the application of structured data and the acquisition, creation, update and comparison of health data. In the prior art, no attention has been yet paid to these problems, nor have the solutions been found to solve them. Therefore, it is an urgency to form comparable and meaningful quantitative indexes on the basis of analyzing the key structure of a single fundus image and the lesions related to various diseases, in the face of massive regular screening results of users and fundus images acquired, to finally present a solution to the comparison and statistics of the fundus images.

### SUMMARY OF THE INVENTION

It is an object of this invention to address difficulties in generating a quantitative index and structured data when processing and analyzing massive fundus images by providing a big data health service method based on remote fundus screening. The specific technical solution is as follows.

A big data health service method based on remote fundus screening, including the steps of: acquiring information to be analyzed sent by a remote terminal agency, the information to be analyzed including fundus images and personal data; pre-interpreting the information to be analyzed, and judging whether the information to be analyzed is qualified; extracting characteristic data from the information to be analyzed if the information to be analyzed is qualified, and forming a structured quantitative index; sorting and analyzing the characteristic data and the quantitative index according to a knowledge calculation model to obtain an analysis conclusion; and storing the information to be analyzed, the characteristic data, the quantitative index, and the analysis conclusion into a pre-designed database.

Further, “pre-interpreting the information to be analyzed, and judging whether the information to be analyzed is qualified” further includes the steps of: judging, through the pre-interpretation, whether the fundus images are real, whether the fundus image is structurally complete, whether the fundus image is clear, and whether one or more of the fundus images are usable; returning relevant qualified information to the remote terminal agency if the information to be analyzed is qualified; returning relevant unqualified information to the remote terminal agency if the information to be analyzed is unqualified, the relevant unqualified information notifying that the remote terminal agency should recollect the information to be analyzed.

Further, “pre-interpreting the information to be analyzed, and judging whether the information to be analyzed is qualified” further includes the step of: sending, by the remote terminal agency, a notification that a user should not leave the remote terminal agency until a notification is returned that the information to be analyzed is qualified, according to a preset rule, before returning a pre-interpretation result to the remote terminal agency.

Further, “pre-interpreting the information to be analyzed, and judging whether the information to be analyzed is qualified” further includes the steps of: returning relevant qualified information to the remote terminal agency if the information to be analyzed is qualified; acquiring, by the remote terminal agency, the relevant qualified information, and notifying whether the user should wait for the analysis conclusion, according to the preset rules.

Further, judging “whether the fundus image is structurally complete” further includes the steps of: identifying and calibrating an optic disc and a macula of the fundus image, judging whether the fundus image includes the optic disc and the macula according to an identification result, judging whether the optic disc and the macula are in a preset area of the fundus image according to a calibration result if the fundus image includes the optic disc and the macula, and determining the fundus image structurally complete if the optic disc and the macula are in the preset area of the fundus image.

Further, “extracting characteristic data from the fundus image, and forming a structured quantitative index” further includes the step of: calculating quantitative parameters of a temporal side of the optic disc and a macula fovea according to the calibrated optic disc and macula.

To solve the technical problem, the invention also provides a big data health service system based on remote fundus screening, and the specific technical solution is as follows.

A big data health service system based on remote fundus screening, including: an fundus image collection module, and a remote analysis center module; wherein the fundus image collection module is connected with the remote analysis center module; the fundus image collection module is used for: acquiring information to be analyzed, the information to be analyzed including: fundus images and personal data, and sending the information to be analyzed to the remote analysis center module; the remote analysis center module is used for: receiving the information to be analyzed, pre-interpreting the information to be analyzed, and judging whether the information to be analyzed is qualified; extracting characteristic data from the information to be analyzed if the information to be analyzed is qualified, and forming a structured quantitative index; sorting and analyzing the characteristic data and the quantitative index according to a knowledge calculation model to obtain an analysis conclusion; and storing the information to be analyzed, the characteristic data, the quantitative index, and the analysis conclusion into a pre-designed database.

Further, pre-interpreting includes: judging whether the fundus images are real, whether the fundus image is structurally complete, whether the fundus image is clear, and whether one or more of the fundus images are usable; the remote analysis center module is further used for returning relevant qualified information to the fundus image collection module if the information to be analyzed is qualified; returning relevant unqualified information to the fundus image collection module if the information to be analyzed is unqualified, the relevant unqualified information notifying that the fundus image collection module should recollect the information to be analyzed.

Further, the fundus image collection module is further used for: sending a notification that a user should not leave the fundus image collection module until a notification is returned that the information to be analyzed is qualified, according to preset rules, before returning a pre-interpretation result to the fundus image collection module.

Further, the remote analysis center module is further used for: returning relevant qualified information to the fundus image collection module if the information to be analyzed is qualified; the fundus image collection module is further used for: acquiring the relevant qualified information, and notifying whether the user should wait for the analysis conclusion, according to the preset rules.

This invention is advantageous in that: the information to be analyzed sent by the remote terminal agency is acquired, wherein the information to be analyzed includes the fundus image and personal data, and is pre-interpreted to judge whether the information to be analyzed is qualified, and a complete closed-loop quality assurance system is formed, which is very important because as such, each piece of information to be analyzed is fully usable, a reliable acquisition of user information is ensured, the user experience is improved, and these all contribute to the final formation of an analyzable and updatable large data base; if the information to be analyzed is qualified, the characteristic data are extracted from the information to be analyzed, and the structured quantitative index is formed; the characteristic data and the quantitative index are stored into the pre-designed database; the characteristic data and the quantitative index are sorted and analyzed according to the knowledge calculation model to obtain the analysis conclusion; and the information to be analyzed, the characteristic data, the quantitative index and the analysis conclusion are stored into the pre-designed database. The above steps can produce the quantitative index and characteristic data with uniform comparability for the final fundus images such processed, no matter what type of fundus camera or which working mode is used; the information to be analyzed, the quantitative index, the characteristic data, and the analysis conclusion are stored in the pre-designed database, so that a whole big data service platform is established, and medical practitioners are facilitated greatly in disease diagnosis and the like.

Further, the information to be analyzed is pre-interpreted, so that the information to be analyzed which is finally subjected to the extraction of the characteristic data can be ensured to be absolutely usable, and a user is saved from the trouble of visiting in person again in the case that the information to be analyzed is found to be not usable by the remote analysis center too late, so the user experience is improved and possible waste of time is avoided; the remote analysis center benefits from this because the usable information to be analyzed not only ensures the stability and accuracy of the diagnosis result, but also improves the diagnosis efficiency and avoids repetitive job.

Further, before the information to be analyzed is qualified, according to preset rules, the remote terminal agency can tell the user not to leave until a notification that the information to be analyzed is qualified is returned, this process avoids a situation that the information to be analyzed is not qualified but the user has left, and thus improves the user experience.

Further, if the fundus image is qualified, characteristic data are extracted from the fundus image, and a structured quantitative index is formed, which includes calculating quantitative parameters of a temporal side of the optic disc and a macula fovea according to the calibrated optic disc and macula. The absolute distance from the temporal side of the optic disc to the macula fovea of a normal person is basically constant, and parameters for subsequent quantitative analysis are acquired according to the given absolute distance from the temporal side of the optic disc to the macula fovea and a diameter of the optic disc; the result data are converted from an absolute representation to a relative representation, and normalized to form meaningful and comparable data. As such, the fundus images from different sources can form meaningful and comparable quantitative indexes, so that all the fundus images can be generally comparable.

### BRIEF DESCRIPTION OF THE DRAWINGS

FIG. 1 is a flow diagram of a big data health service method based on remote fundus screening in accordance with an embodiment of the present invention;

FIG. 2 is a block diagram of a big data health service system based on remote fundus screening in accordance with an embodiment of the present invention.

### DESCRIPTION OF REFERENCE SIGNS

200. Storage device.

### DETAILED DESCRIPTION OF THE INVENTION

Reference is made to the specific embodiments and accompanying drawings to explain the technical aspects, structural features, objects and effects of the technical solutions in detail.

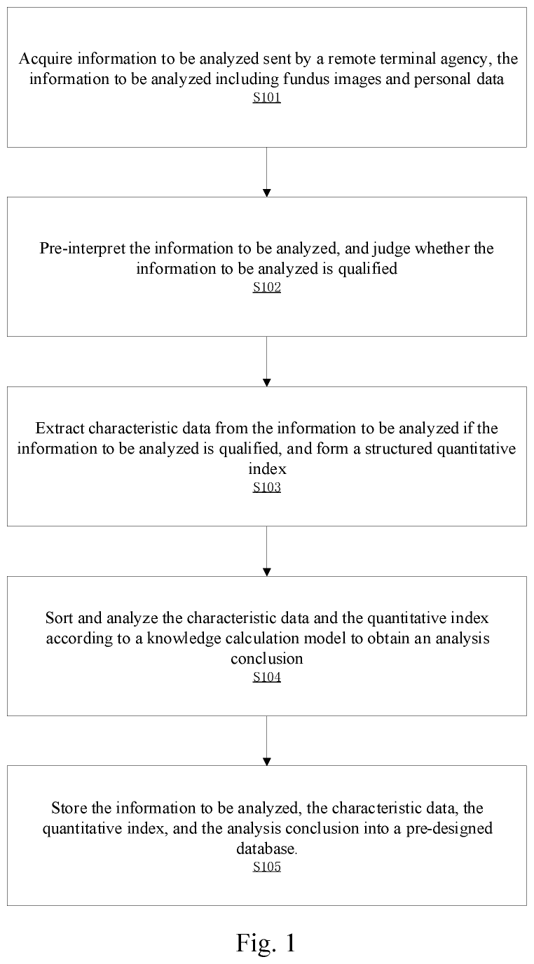

Referring to FIG. 1, in the present embodiment, some or all of the steps in a big data health service method based on remote fundus screening may be performed by programming to instruct relevant hardware, the program may be stored in a storage medium readable by a computer device, for performing some or all of the steps of the embodiments described below. The computer devices include, but are not limited to: personal computers, servers, general-purpose computers, special-purpose computers, network equipment, intelligent mobile terminals, intelligent household equipment, and wearable intelligent equipment; the storage media include, but are not limited to: RAM, ROM, mobile hard disk, network server storage, and network cloud storage.

In the embodiment, the big data health service method based on remote fundus screening is implemented specifically as follows.

Step S101: information to be analyzed sent by a remote terminal agency is acquired, the information to be analyzed includes fundus images and personal data. This step can be implemented as follows: the remote terminal agency is provided with a fundus image collection terminal and any computer device capable of receiving and sending information, such as a PC, the fundus image is acquired through the fundus image collection terminal, then the fundus image is transmitted to the PC, the personal data are also input into the PC, and the fundus image and the personal data are sent together to a remote analysis center by the PC.

In other embodiments, the remote terminal agency (such as a community medical clinic) collects a fundus image of User A with a fundus camera, the fundus camera transmits the fundus image to a computer of a terminal application agency through a universal serial bus, meanwhile, personal information or personal data can be input into a computer that transmits the information to a remote interpretation center.

In this embodiment, the personal data or medical records include one or more of name, identity card, height, weight, waistline, family genetic history, medication, blood glucose, blood pressure, eyesight, fitness, diet, living habits, and history of smoking or drinking.

After the information to be analyzed is obtained, step S102 is performed: the information to be analyzed is pre-interpreted to judge whether the information to be analyzed is qualified. This step can be implemented as follows: the pre-interpretation includes judging whether the fundus images are real, whether the fundus image is structurally complete, whether the fundus image is clear, and whether one or more of the fundus images are usable

This step can be implemented as follows: the pre-interpretation information input by a quality inspector is acquired; whether the fundus image is qualified is determined according to the pre-interpretation information input by the quality inspector in conjunction with a pre-interpretation result of automatic fundus image analysis; the input information includes a quality grade of the fundus image. The fundus image can be automatically analyzed, for example, by training an SVM model based on the images collected previously and graded by a professional doctor, so that the model can grade the images as per their quality. Therefore, when a fundus image is collected, on one hand, the trained SVM model is used for judging on the fundus image, and on the other hand, the quality inspector, for example, a professional ophthalmologist, inputs information on the quality grade of the fundus image. The fundus image is pre-interpreted by combining them both, human assistance can avoid errors of automatic analysis, and in turn, automatic analysis can reduce workload and complexity of human recognition. The fundus image disqualified by the SVM model is rechecked by the human to avoid mistakes in judgment, thereby ensuring that the fundus image is absolutely usable at last.

In other embodiments, the fundus image analysis may be automated without the participation of quality inspectors.

Further, upon reception of a fundus image, whether the fundus image is real is subjected to judgment, and if the fundus image is not real, it may be sent by mistake, then the current interpretation is directly terminated, a corresponding notification is returned to the remote terminal agency, telling that the acquired fundus image is not real, and the user shall recollect the fundus image. If the fundus image is real, then whether the fundus image is structurally complete is subjected to judgment by identifying and calibrating an optic disc and a macula of the fundus image, judging whether the fundus image includes the optic disc and the macula according to an identification result, judging whether the optic disc and the macula are in a preset area of the fundus image according to a calibration result if the fundus image includes the optic disc and the macula, and determining the fundus image structurally complete if the optic disc and the macula are in the preset area of the fundus image.

The implementation specifically includes green channel selection, median filtering, limited contrast enhancement and gray normalization on the fundus image to be inspected. By preprocessing the fundus image, redundant background in the fundus image can be removed, noise is effectively removed, and subsequent fundus image analysis is facilitated. Specifically, in any colored fundus image, there is much noise in the blue channel, useful information is basically lost; the macula is more prominent in the red channel, and information is lost on dark blood vessels, micro hemangiomas and the like, hence the colored fundus image to be inspected is subjected to green channel selection in the embodiment, and fundus blood vessels are reserved and highlighted to the greatest extent. In order to remove the noise and keep the boundary information well, the fundus image in the green channel is subjected to median filtering in the embodiment, so that the noise is removed; to obtain a better blood vessel extraction effect, the denoised image is subjected to contrast enhancement. To avoid over-brightness after image enhancement, a limited contrast enhancement, namely, CLAHE, is adopted in the embodiment. Finally, normalization is conducted to enable pixel values of all pixels in one image to fall between 0 and 1.

A binarized blood vessel image is extracted from the preprocessed fundus image through the OTSU algorithm, and the binarized blood vessel image is corroded through a morphological method to obtain a main blood vessel. Specifically, a threshold value is calculated for the preprocessed fundus image through the OTSU algorithm, and the pixels with a gray value larger than the threshold value are identified as a blood vessel according to the following formula;

$${Map}_{v} ( i , j ) = \{ \begin{matrix}1 , & i f Gv ( i , j ) > T \\ 0 , & otherwise\end{matrix}$$

Structural elements are such configured that the diameter of the optic disc is ⅛-⅕ of the width of the image and the width of the main blood vessel is ¼ of the diameter of the optic disc, the extracted blood vessel is corroded with the structural elements, minimal blood vessels are removed, and the main blood vessel is obtained. Given the main blood vessel, the main blood vessel is subjected to a parabolic fitting calculation, and the center of the optic disc is positioned according to the calculation result. Specifically, a coordinate system is established by taking the upper left corner of the fundus image as an original point, the horizontal direction as an X axis, and the vertical direction as a Y axis; each pixel in the main blood vessel is mapped to have coordinates in the coordinate system;

As shown in the following formula, the main vessel is parabolically fitted according to the least square method, the parameters of the parabola are determined, and the vertex of the parabola is figured out.

f(x)=ax²+bx+c

S(a,b,c)=Σᵢ₌₁^{N}|f(xᵢ)−yᵢ|²

Whether the parabolic vertex falls in the original fundus image is subjected to judgment, and if the parabolic vertex falls in the original fundus image, the parabolic vertex is defined as the center of the optic disc. The macula is positioned on the basis of appearance features and structural features: according to the positional relationship between the macula and the optic disc, a range for searching the fovea is further reduced on the basis of the determined center of the optic disc. In one preferred manner, since the distance between the macula fovea and the center of the optic disc is generally 2 to 3 times the diameter of the optic disc, so an annular mask is constructed with the center of the optic disc as the center of the circle, and the annular mask is defined as the fovea search range; and then, in the search range, the fovea is positioned given that the brightness of the fovea is the lowest. In a preferred manner, a fast searching based on brightness comparison among regions is adopted to determine the position of the fovea; and finally, given the brightness information, the macula area is fitted with a circle with the fovea as the circle center.

Given the macula and the center of the optic disc, the structural integrity of the fundus image is subjected to judgment. An image satisfying the judgment conditions shown in Table 1 is an image whose integrity is qualified. Herein, Dod is the optic disc diameter.

TABLE 1

| Different | | |
|---|---|---|
| distances | Description | Judgment condition |
| D_{OD-FOVEA} | Distance from | 1.5D_{OD} < DO_{D-FOVEA} < |
| | the macula fovea to | 3.5D_{OD} |
| | the center of the optic | |
| | disc | |
| D_{OD-EDGE} | Center-to-edge | 1D_{OD} < D_{OD-EDGE} |
| | distance of optic disc | |
| D_{FOVEA-EDGE} | Center-to-edge | 2.5D_{OD} < D_{FOVEA-EDGE} |
| | distance of macula | |

If the fundus image is real, whether the fundus image structure is clear is subjected to judgment, specifically, whether the small blood vessels on the surface of the optic disc and the retinal nerve fiber layer of the posterior pole of the fundus image are distinguishable is subjected to judgment, and if the small blood vessels on the surface of the optic disc and the retinal nerve fiber layer of the posterior pole of the fundus image are distinguishable, the definition of the fundus image is qualified. The implementation includes the following steps:

a, given the identified optic disc center and macula fovea, a region is defined as a region of interest 1 with the optic disc as the circle center and ranging 1.5 times the diameter of the optic disc, and a region of interest 2 is defined with the macula fovea as the circle center and ranging 1 times the diameter of the optic disc;

b, a definition evaluation operator is selected based on the defined region of interest 1 and the region of interest 2, a definition evaluation value is calculated, and then the definition evaluation is completed.

Step S103: if the information to be analyzed is qualified, the characteristic data are extracted from the information to be analyzed, and a structured quantitative index is formed, specifically, by calculating quantitative parameters of a temporal side of the optic disc and a macula fovea according to the calibrated optic disc and macula. The temporal coordinates (ODX, ODY) of the optic disc are calculated according to the coordinates of the center of the optic disc and the radius of the optic disc; the absolute distance between the temporal side of the optic disc and the macula fovea is calculated according to the temporal side coordinates of the optic disc and the macula fovea coordinates, and a Euclidean distance between the temporal side of the optic disc and the macula fovea is calculated according to the following formula to serve as the absolute distance between the optic disc center and the macula fovea in the image;

OMD=\sqrt{|ODX−MX|²+|ODY−MY|²} Formula 2

where all coordinate values take the upper left corner pixel of the fundus image as an origin.

c, the macula fovea is generally about 3 mm away from the temporal side edge of the optic disc, so a standard d for subsequent quantitative analysis is obtained according to the given absolute distance from the temporal side of the optic disc to the macula fovea and the diameter of the optic disc according to the following formula:

d=DMD−ODD Formula 3

In this embodiment, the data obtained is converted from an absolute representation to a relative representation on the scale of d, and normalized to form meaningful and comparable data.

In this embodiment, if hard exudation has been detected, and the Euclidean distance Di of each hard exudation to the macula fovea has been calculated, then normalization may be performed according to Formula 1. On this basis, the standard minimum distance from the hard exudation to the macula fovea is obtained.

$${d}_{i}^{′} = \frac{{d}_{i}}{d} \qquad Formula 4$$

Step S104: the characteristic data and the quantitative index are sorted and analyzed according to the knowledge calculation model to obtain an analysis conclusion. Step S105: the information to be analyzed, the characteristic data, the quantitative index and the analysis conclusion are stored into the pre-designed database.

Specifically, the fundus camera technology for diabetic retinopathy (DR) screening has matured, and DR screening has prevention and treatment guidelines and diagnostic standards that are related to diabetes and can guide treatment. Eyes are the only parts of the body where blood vessels and nerves can be directly seen without surgery. Medical evidence shows that the circulatory system of the retina and the brain have similar anatomical, physiological and embryonic development characteristics. Therefore, through the fundus blood vessels, we can understand the severity of diseases of the whole body, especially of the cerebral arteries and the middle and small arteries in the whole body; according to guidelines for the prevention and treatment of hypertension in China, retinal artery disease can reflect the condition of small vessel disease. If we can find out the key methods for quantitative analysis through regular screening and comparison of fundus images, we can analyze quantitatively, gather statistics and compare the retinopathy characteristics or vascular changes of fundus images collected from different people, or from the same person but at different times or with different devices, so as to form structured health data; through the “knowledge calculation model”, and the “disease early warning and health assessment engine” established on the basis of the “knowledge calculation model”, it is possible to provide early warning or abnormality screening for diseases such as diabetic retinopathy, diabetic nephropathy, hypertension, and stroke; in particular, if diabetic retinopathy patients do not develop an appropriate lifestyle to intervene basic therapy and drug treatment, their fundus retinopathy characteristics or condition will definitely continue to get worse. In view of this, the “knowledge calculation model” is established or relied upon to provide statistics, calculation and analysis methods for fundus images of blood vessel characteristics, which is of great significance for the timely detection of fundus retinopathy and fundus vascular changes and other characteristics, provision of auxiliary diagnostic information or health management and service suggestions, and the development of big data health services.

Therefore, according to the characteristics extracted from the user's fundus image and the necessary personal data, a structured quantitative index and a highly professional “knowledge base” are formed. The quantitative index includes: health information such as past medical history, height, weight, waistline, fitness and diet, history of diabetes and previous treatment, and history of hypertension and previous treatment; personal information such as family genetic history and living habits; medical records; and DR interpretation results related to the number, area and location of microvascular tumors, bleeding sites, hard exudation, cotton wool spots, etc. The quantitative index further includes whether there are proliferated blood vessels, whether there is macular edema; arteriovenous ratio, arterial diameter narrowing, arteriovenous cross, indentation and position records, gold or silver wire arteries in the region of interest; changes of one or more blood vessels and nerve fiber layer changes, etc. Therefore, relying on the “knowledge calculation model”, it is possible to provide a method of statistics, calculation and analysis of fundus retinopathy, vascular changes and other characteristics, namely, the “disease early warning and health assessment engine”.

The information to be analyzed sent by the remote terminal agency is acquired, wherein the information to be analyzed includes fundus images and personal data; the information to be analyzed is pre-interpreted to judge whether the information to be analyzed is qualified; if the information to be analyzed is qualified, the characteristic data are extracted from the information to be analyzed, and the structured quantitative index is formed; the characteristic data and the quantitative index are stored into the pre-designed database; the characteristic data and the quantitative index are sorted and analyzed according to the knowledge calculation model to obtain the analysis conclusion; and the information to be analyzed, the characteristic data, the quantitative index and the analysis conclusion are stored into the pre-designed database. The above steps can produce the quantitative index and characteristic data with uniform comparability for the final fundus images such processed, no matter what type of fundus camera or which working mode is used; the information to be analyzed, the quantitative index, the characteristic data, and the analysis conclusion are stored in the pre-designed database, so that a whole big data service platform is established, and medical practitioners are facilitated greatly in disease diagnosis and the like. Further, the information to be analyzed is pre-interpreted, so that the information to be analyzed which is finally subjected to the extraction of the characteristic data can be ensured to be absolutely usable, and a user is saved from the trouble of visiting in person again in the case that the information to be analyzed is found to be not usable by the remote analysis center too late, so the user experience is improved and possible waste of time is avoided; the remote analysis center benefits from this because the usable information to be analyzed not only ensures the stability and accuracy of the diagnosis result, but also improves the diagnosis efficiency and avoids repetitive job. Further, before the information to be analyzed is qualified, according to preset rules, the remote terminal agency can tell the user not to leave until a notification that the information to be analyzed is qualified is returned, this process avoids a situation that the information to be analyzed is not qualified but the user has left, and thus improves the user experience. Further, if the fundus image is qualified, characteristic data are extracted from the fundus image, and a structured quantitative index is formed, which includes calculating quantitative parameters of a temporal side of the optic disc and a macula fovea according to the calibrated optic disc and macula. The absolute distance from the temporal side of the optic disc to the macula fovea of a normal person is basically constant, and parameters for subsequent quantitative analysis are acquired according to the given absolute distance from the temporal side of the optic disc to the macula fovea and a diameter of the optic disc; the result data are converted from an absolute representation to a relative representation, and normalized to form meaningful and comparable data. As such, the fundus images from different sources can form meaningful and comparable quantitative indexes, so that all the fundus images can be generally comparable.

It should be noted that in other embodiments, the optic disc and macula may also be positioned manually.

In the embodiment, before the step of “sending the information to be analyzed to the remote analysis center”, the method further includes the following steps: the remote terminal agency is provided with specific software which can be used for pre-interpreting the fundus image and the personal data offline, and if they are judged to be qualified, a corresponding notification is sent that the user may leave or continue to stay nearby waiting for analysis results from the remote analysis center.

Further, “pre-interpreting the information to be analyzed, and judging whether the information to be analyzed is qualified” further includes the step of: sending, by the remote terminal agency, a notification that a user should not leave the remote terminal agency until a notification is returned that the information to be analyzed is qualified, according to a preset rule, before returning a pre-interpretation result to the remote terminal agency. Specifically, if the remote terminal agency is not provided with software that can be used for pre-interpreting the fundus image and personal data offline, then according to the preset rules (i.e., whether the remote terminal agency serves for or is closely related to the protocols of the remote analysis center, and when it is not necessary to buy specific software, the agreement on the process or quality control system is followed), the remote terminal agency informs the user of not leaving the remote terminal agency until it's notified that the information to be analyzed is found qualified before returning the pre-interpretation result to the remote terminal agency; the user may also be allowed to stay nearby waiting for remote interpretation results from a remote interpretation center.

Further, “pre-interpreting the information to be analyzed, and judging whether the information to be analyzed is qualified” further includes the steps of: returning relevant qualified information to the remote terminal agency if the information to be analyzed is qualified; acquiring, by the remote terminal agency, the relevant qualified information, and notifying whether the user should wait for the analysis conclusion, according to the preset rules. Specifically, after the remote terminal agency receives the notification that the information to be analyzed is qualified, the user can be told whether to wait until the analysis conclusion is available according to the actual situation.

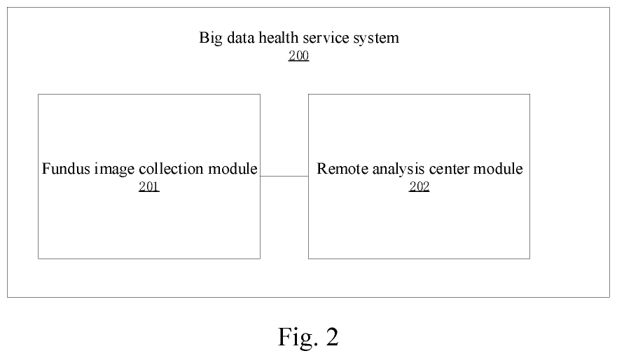

Referring to FIG. 2, in the present embodiment, the fundus image collection module 201 includes at least a fundus image collection camera and a computer; the remote analysis center module 202 may be a storage device. A corresponding remote analysis center APP is installed on the storage device, or a corresponding remote analysis center website is directly opened, so that information to be analyzed transmitted by the fundus image collection module 201 can be processed. An embodiment of a big data health service system 200 based on remote fundus screening is as follows.

A big data health service system 200 based on remote fundus screening, including: an fundus image collection module 201, and a remote analysis center module 202; wherein the fundus image collection module 201 is connected with the remote analysis center module 202; the fundus image collection module 201 is used for: acquiring information to be analyzed, the information to be analyzed including: fundus images and personal data, and sending the information to be analyzed to the remote analysis center module 202; the remote analysis center module 202 is used for: receiving the information to be analyzed, pre-interpreting the information to be analyzed, and judging whether the information to be analyzed is qualified; extracting characteristic data from the information to be analyzed if the information to be analyzed is qualified, and forming a structured quantitative index; sorting and analyzing the characteristic data and the quantitative index according to a knowledge calculation model to obtain an analysis conclusion; and storing the information to be analyzed, the characteristic data, the quantitative index, and the analysis conclusion into a pre-designed database. Further, pre-interpreting includes: judging whether the fundus images are real, whether the fundus image is structurally complete, whether the fundus image is clear, and whether one or more of the fundus images are usable; the remote analysis center module 202 is further use for returning relevant qualified information to the fundus image collection module 201 if the information to be analyzed is qualified; returning relevant unqualified information to the fundus image collection module 201 if the information to be analyzed is unqualified, the relevant unqualified information notifying that the fundus image collection module 201 should recollect the information to be analyzed.

Further, the fundus image collection module 201 is further used for: sending a notification that a user should not leave the fundus image collection module 201 until a notification is returned that the information to be analyzed is qualified, according to preset rules, before returning a pre-interpretation result to the fundus image collection module 201.

Further, the remote analysis center module 202 is further used for: returning relevant qualified information to the fundus image collection module 201 if the information to be analyzed is qualified; the fundus image collection module 201 is further used for: acquiring the relevant qualified information, and notifying whether the user should wait for the analysis conclusion, according to the preset rules.

The big data health service system 200 based on remote fundus screening acquires information to be analyzed through the fundus image collection module 201, wherein the information to be analyzed includes fundus images and personal data. The information to be analyzed is pre-interpreted by the remote analysis center module 202 to judge whether the information to be analyzed is qualified; if the information to be analyzed is qualified, the characteristic data are extracted from the information to be analyzed, and the structured quantitative index is formed; the characteristic data and the quantitative index are sorted and analyzed according to the knowledge calculation model to obtain the analysis conclusion; and the information to be analyzed, the characteristic data, the quantitative index and the analysis conclusion are stored into the pre-designed database. The above functional modules can produce the quantitative index and characteristic data with uniform comparability for the final fundus images such processed, no matter what type of fundus camera or which working mode is used; the information to be analyzed, the quantitative index, the characteristic data, and the analysis conclusion are stored in the pre-designed database, so that a whole big data service platform is established, and medical practitioners are facilitated greatly in disease diagnosis and the like.

Further, the information to be analyzed is pre-interpreted, so that the information to be analyzed which is finally subjected to the extraction of the characteristic data can be ensured to be absolutely usable, and a user is saved from the trouble of visiting in person again in the case that the information to be analyzed is found to be not usable by the remote analysis center too late, so the user experience is improved and possible waste of time is avoided; the remote analysis center benefits from this because the usable information to be analyzed not only ensures the stability and accuracy of the diagnosis result, but also improves the diagnosis efficiency and avoids repetitive job.

Further, before the information to be analyzed is qualified, according to preset rules, the remote terminal agency can tell the user not to leave until a notification that the information to be analyzed is qualified is returned, this process avoids a situation that the information to be analyzed is not qualified but the user has left, and thus improves the user experience.

Further, if the fundus image is qualified, characteristic data are extracted from the fundus image, and a structured quantitative index is formed, which includes calculating quantitative parameters of a temporal side of the optic disc and a macula fovea according to the calibrated optic disc and macula. The absolute distance from the temporal side of the optic disc to the macula fovea of a normal person is basically constant, and parameters for subsequent quantitative analysis are acquired according to the given absolute distance from the temporal side of the optic disc to the macula fovea and a diameter of the optic disc; the result data are converted from an absolute representation to a relative representation, and normalized to form meaningful and comparable data. As such, the fundus images from different sources can form meaningful and comparable quantitative indexes, so that all the fundus images can be generally comparable.

It should be noted that although the above embodiments have been described herein, the scope of the present invention is not limited thereto. Therefore, on the basis of innovative concept of the present invention, changes and modifications to the embodiments described herein, or equivalent structure or equivalent process transformations made by using the description and drawings of the present invention, direct or indirect application of the above technical solutions to other related technical fields, shall fall within the scope of the present invention.

What is claimed is:

1. A big data health service method based on remote fundus screening, characterized by comprising the steps of: acquiring information to be analyzed sent by a remote terminal agency, the information to be analyzed comprising fundus images and personal data; pre-interpreting the information to be analyzed, and judging whether the information to be analyzed is qualified; extracting characteristic data from the information to be analyzed if the information to be analyzed is qualified, and forming a structured quantitative index; sorting and analyzing the characteristic data and the quantitative index according to a knowledge calculation model to obtain an analysis conclusion; and storing the information to be analyzed, the characteristic data, the quantitative index, and the analysis conclusion into a pre-designed database.

2. The big data health service method based on remote fundus screening according to claim 1, characterized in that “pre-interpreting the information to be analyzed, and judging whether the information to be analyzed is qualified” further comprises the steps of: judging, through the pre-interpretation, whether the fundus images are real, whether the fundus image is structurally complete, whether the fundus image is clear, and whether one or more of the fundus images are usable; returning relevant qualified information to the remote terminal agency if the information to be analyzed is qualified; returning relevant unqualified information to the remote terminal agency if the information to be analyzed is unqualified, the relevant unqualified information notifying that the remote terminal agency should recollect the information to be analyzed.

3. The big data health service method based on remote fundus screening according to claim 1, characterized in that “pre-interpreting the information to be analyzed, and judging whether the information to be analyzed is qualified” further comprises the step of: sending, by the remote terminal agency, a notification that a user should not leave the remote terminal agency until a notification is returned that the information to be analyzed is qualified, according to preset rules, before returning a pre-interpretation result to the remote terminal agency.

4. The big data health service method based on remote fundus screening according to claim 1, characterized in that “pre-interpreting the information to be analyzed, and judging whether the information to be analyzed is qualified” further comprises the steps of: returning relevant qualified information to the remote terminal agency if the information to be analyzed is qualified; acquiring, by the remote terminal agency, the relevant qualified information, and notifying whether the user should wait for the analysis conclusion, according to the preset rules.

5. The big data health service method based on remote fundus screening according to claim 2, characterized in that
judging “whether the fundus image is structurally complete” further comprises the steps of: identifying and calibrating an optic disc and a macula of the fundus image, judging whether the fundus image comprises the optic disc and the macula according to an identification result, judging whether the optic disc and the macula are in a preset area of the fundus image according to a calibration result if the fundus image comprises the optic disc and the macula, and determining the fundus image structurally complete if the optic disc and the macula are in the preset area of the fundus image.

6. The big data health service method based on remote fundus screening according to claim 5, characterized in that “extracting characteristic data from the fundus image, and forming a structured quantitative index” further comprises the step of: calculating quantitative parameters of a temporal side of the optic disc and a macula fovea according to the calibrated optic disc and macula.

7. A big data health service system based on remote fundus screening, characterized by comprising: a fundus image collection module, and a remote analysis center module; wherein the fundus image collection module is connected with the remote analysis center module; the fundus image collection module is used for: acquiring information to be analyzed, the information to be analyzed comprising: fundus images and personal data, and sending the information to be analyzed to the remote analysis center module; the remote analysis center module is used for: receiving the information to be analyzed, pre-interpreting the information to be analyzed, and judging whether the information to be analyzed is qualified; extracting characteristic data from the information to be analyzed if the information to be analyzed is qualified, and forming a structured quantitative index; sorting and analyzing the characteristic data and the quantitative index according to a knowledge calculation model to obtain an analysis conclusion; and storing the information to be analyzed, the characteristic data, the quantitative index, and the analysis conclusion into a pre-designed database.

8. The big data health service system based on remote fundus screening according to claim 7, characterized in that pre-interpreting comprises: judging whether the fundus images are real, whether the fundus image is structurally complete, whether the fundus image is clear, and whether one or more of the fundus images are usable; the remote analysis center module is further used for returning relevant qualified information to the fundus image collection module if the information to be analyzed is qualified; returning relevant unqualified information to the fundus image collection module if the information to be analyzed is unqualified, the relevant unqualified information notifying that the fundus image collection module should recollect the information to be analyzed.

9. The big data health service system based on remote fundus screening according to claim 7, characterized in that the fundus image collection module is further used for: sending a notification that a user should not leave the fundus image collection module until a notification is returned that the information to be analyzed is qualified, according to preset rules, before returning a pre-interpretation result to the fundus image collection module.

10. The big data health service system based on remote fundus screening according to claim 7, characterized in that the remote analysis center module is further used for: returning relevant qualified information to the fundus image collection module if the information to be analyzed is qualified; the fundus image collection module is further used for: acquiring the relevant qualified information, and notifying whether the user should wait for the analysis conclusion, according to the preset rules.
